(19) United States
(12) Patent Application Publication
(10) Pub. No.: US 2015/0351798 A1
(43) Pub. Date: Dec. 10, 2015

(54) Needle Aponeurotomy Apparatus and Method

(71) Applicant: Cedar Farms Medical, LLC, Collierville, TN (US)

(72) Inventors: William L. Bourland, Memphis, TN (US); Eric O. Cohea, US, TN (US)

(73) Assignee: Cedar Farms Medical, LLC, Collierville, TN (US)

(21) Appl. No.: 14/298,963

(22) Filed: Jun. 9, 2014

(51) Int. Cl. A61B 17/34

(52) U.S. Cl. CPC A61B 17/3476; A61B 17/3417

(57) ABSTRACT

A needle aponeurotomy apparatus for performing needle aponeurotomy upon a Dupuytren's band in a patient's hand and a method of use. The apparatus comprises a handheld body, a first needle having a longitudinal bore and fixedly mounted to the body, and a second needle received into the bore of the first needle. A motor reciprocates the second needle with respect to the body. An adjustment is provided so that the tip of the second needle maximally extends to a certain distance, about 2.5 to 3.0 mm, beyond the distal end of the first needle during each reciprocation. During treatment, the first needle is inserted into a patient's hand and the motor is caused to reciprocate the second needle, which repeatedly penetrates and thus weakens a Dupuytren's band of the patient.

### CROSS REFERENCE TO RELATED APPLICATIONS

Not applicable.

### STATEMENT REGARDING FEDERALLY SPONSORED RESEARCH OR DEVELOPMENT

Not applicable.

### REFERENCE TO COMPACT DISC(S)

Not applicable.

### BACKGROUND OF THE INVENTION

1. Field of the Invention

The present invention relates, in general, to hand surgery, and in particular, to surgical apparatus and methods for treatment of Dupuytren's contracture of the hand.

2. Information Disclosure Statement

Dupuytren's contracture of the hand, or Dupuytren's disease, is a hereditary condition found mostly in adult men of Northern European descent. It is an abnormal thickening of the tissue of the hand just beneath the skin. This thickening occurs in the palm and can extend into the fingers, and usually begins as a nodule that can then form a cord. Over time, the cord can form a contracture (“Dupuytren's contracture”), thereby bending the fingers into a flexed position. Although the skin may become involved in the process, the deeper structures, such as the tendons, are not directly involved. Occasionally, the disease will cause thickening on top of the finger knuckles, or cause lumps or cords within the soles of the feet (“planar fibromatosis”). As the cord thickens and the fingers are drawn into the palms, hand function can become reduced.

There are presently three primary prior art forms of treatment of Dupuytren's disease in the United States. The oldest treatment is to make an open excision, involving either a Z-Plasty or zig-zag incision, in the palm of the hand, and the diseased tissue is then removed from the patient's palm. The disadvantage of treatment using this manner of treatment is that an extensive recovery time, including months of rehabilitation therapy to restore function in the hand, is usually required.

A more current technique of treatment is by injection of a collagenase enzyme (Xiaflex) that has been on the market for about three years. The collagenase is injected into the Dupuytren's cord, which causes the cord to dissolve and become weakened over a period of weeks, after which the cord can then be manipulated and broken by the doctor as the fingers are straightened, thereby reducing and eliminating the contracture of the fingers. The disadvantage of using this manner of treatment is that there are notable side effects, including the possibility of tendon rupture and tendon damage, tendon sheath damage, and nerve damage, because the dissolving effect of the collagenase is not limited to the Dupuytren's cord, and often repair surgery becomes necessary.

The third most common treatment in the United States is a needle aponeurotomy (“NA”), also called needle aponevrotomy or percutaneous needle fasciotomy (“PNF”). This technique was developed in France about thirty years ago, and involves manually taking a small gauge needle and repeatedly penetrating the Dupuytren's cord or band multiple times to weaken the cord, and then mechanically stretching and then breaking the cord or band by straightening out the fingers into a normal position, typically with a characteristic snap. Needle aponeurotomy has the advantage of being a non-surgical, ambulant, outpatient procedure. Typically a 25 gauge needle is used to perform the needle aponeurotomy technique, and the doctor can use a local block anesthesia to relieve the pain of the procedure while repeatedly penetrating the Dupuytren's cord. Use of a local block anesthesia rather than a general anesthesia allows the doctor to monitor if/when the medial nerve, which runs alongside the Dupuytren's cord, becomes hit by the needle. The advantage of the needle aponeurotomy technique is that there is not the significant recovery period that is required by the Z-Plasty technique, and the damage to tendons and nerves caused by collagenase enzyme injection does not occur. The disadvantage of needle aponeurotomy is that it is time consuming and tiring for a doctor to repeatedly penetrate the Dupuytren's cord until it becomes weakened, and the penetration depth of the needle into the Dupuytren's cord varies with each penetration.

It should be noted that, despite successful treatment of Dupuytren's disease with any of these treatment methods, the disease is recurrent and can also extend into other fingers, and not all of the thickening cords or bands may be apparent at the original time of treatment.

It is therefore desirable to have an apparatus and minimally invasive method for performing needle aponeurotomy for treatment of Dupuytren's contracture of the hand, such that the needle penetration of the Dupuytren's cord is of a controlled depth and at a faster rate than heretofore possible with the prior art.

### BRIEF SUMMARY OF THE INVENTION

The present invention is an apparatus for performing needle aponeurotomy and a minismally-invasive method of using the needle aponeurotomy apparatus to weaken a Dupuytren's cord in a patient's hand so that the Dupuytren's cord can be broken. The apparatus comprises a handheld body, a first needle having a longitudinal bore and fixedly mounted to the body, and a second needle received into the bore of the first needle. A motor reciprocates the second needle with respect to the body. An adjustment is provided so that the tip of the second needle maximally extends to a certain distance, about 2.5 to 3.0 mm, beyond the distal end of the first needle during each reciprocation. During treatment, the first needle is inserted into a patient's hand and the motor is caused to reciprocate the second needle, which repeatedly penetrates and thus weakens a Dupuytren's band of the patient. The contracted finger is then forcibly extended, thereby causing the weakened Dupuytren's band to “snap” and break.

It is an object of the present invention to provide an improved needle aponeurotomy apparatus and method of use that is minimally invasive and that is faster than prior art needle aponeurotomy procedures. It is a further object of the present invention to provide an improved needle aponeurotomy apparatus that provides a better result than heretofore possible because the needle penetration of the Dupuytren's cord is of a controlled depth.

### BRIEF DESCRIPTION OF THE SEVERAL VIEWS OF THE DRAWING

FIG. 1 is a perspective view of the present invention assembled and with the protective needle cover over the first and second needles.

FIG. 2 is a perspective view of the present invention assembled, similar to FIG. 1, but with the protective needle cover removed and also showing, in dotted outline, the adjustable movement of the grip to vary the maximal extension distance of the second needle.

FIG. 3 is a sectional view of the grip of the present invention.

FIG. 4 is a side view of the adapter of the present invention.

FIG. 5 is a sectional view of the first needle, Luer lock, and needle safety cover of the present invention.

FIG. 6 is a top view of the linkage and second needle of the present invention.

FIG. 7 is a view showing the procedure of the present invention.

FIG. 8 is a sectional view showing the second needle penetrating a Dupuytren's band D, with an extended position of the second needle shown in dotted outline.

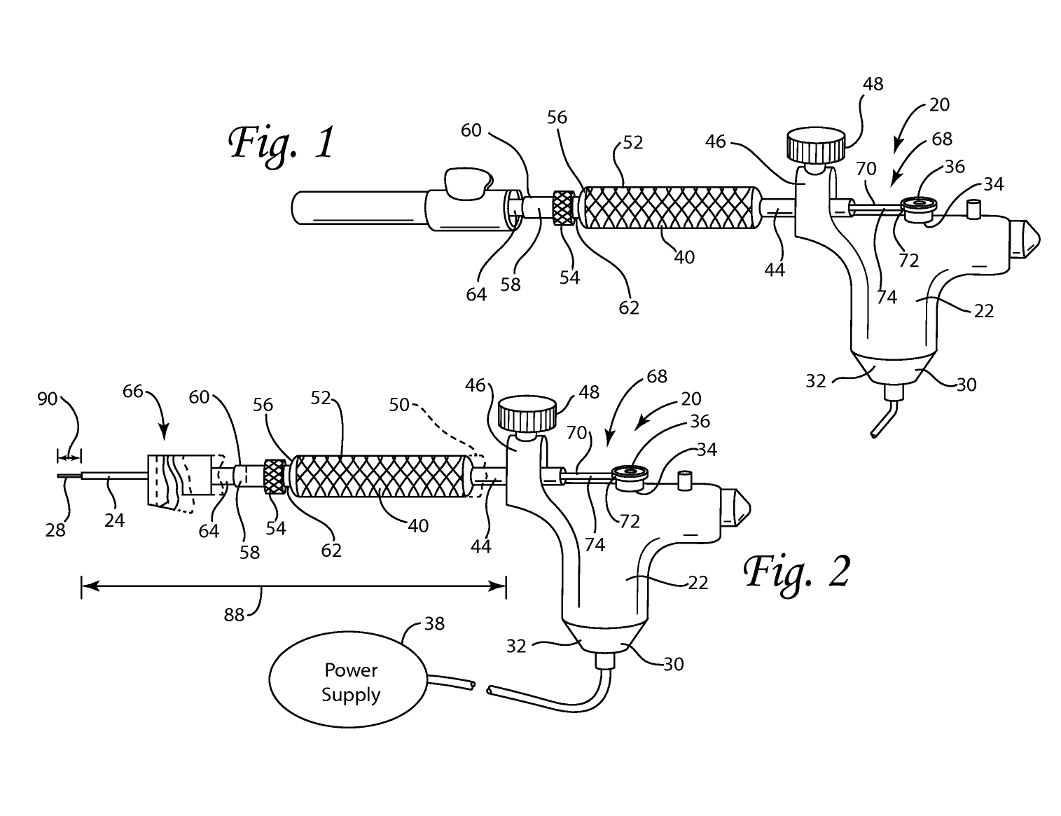

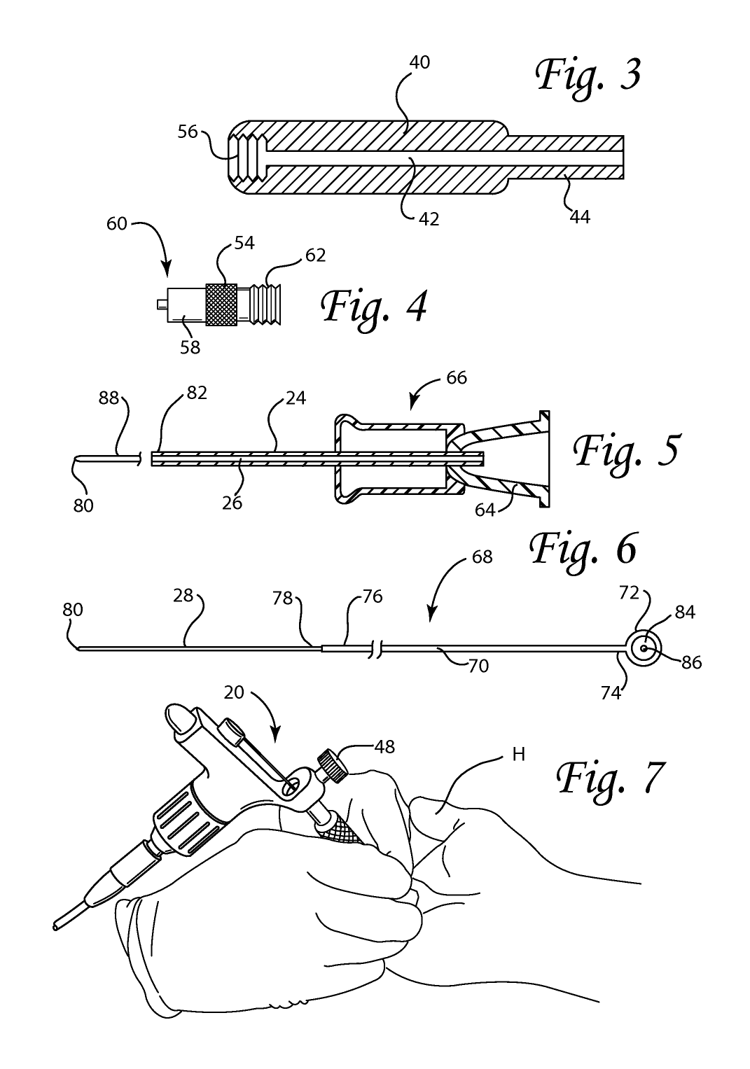

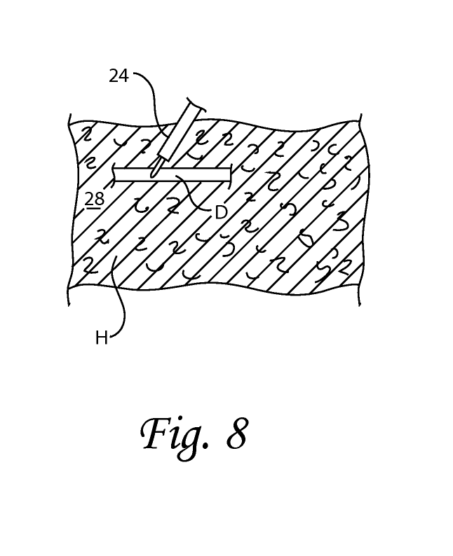

### DETAILED DESCRIPTION OF THE INVENTION

Referring to the figures of the drawings, the preferred embodiment of the needle aponeurotomy apparatus 20 of the present invention is seen to have a handheld body 22 that is held by a doctor while performing a needle aponeurotomy procedure on a Dupuytren's band or cord in a patient's hand H. It should be understood that the terms “Dupuytren's band” and “Dupuytren's cord” are to be understood to be used interchangeably herein to refer to the structure of the thickened tissue of the hand that develops in a patient with Dupuytren's disease. Apparatus 20 includes a first needle 24 having a first longitudinal bore 26 therethrough, and also includes a second needle 28 received into bore 26 for reciprocation therein.

Body 22 includes motor means 30 for reciprocating a second needle 28 with respect to body 22. An acceptable body 22 with motor means 30 is the well-known Spektra Halo Ninja model tattooing gun sold by Fallen Kings Iron, Co., 1819 NW 79th Ave., Doral, Fla. 33126, U.S.A., that is modified by discarding the tattooing apparatus (“cartridge grip”) that is typically sold with that tattooing gun. Motor means 30 includes an electrical motor 32 having a shaft 34 with an offset crankarm 36 that causes the second needle 28 to reciprocate with respect to body 22 in a manner hereinafter described. Motor 32 is connected to a well-known power supply 38 that may have a footswitch (not shown) interposed between power supply 38 and motor 32 so that the motor 32 may be turned on and off as desired. The reciprocation stroke of crankarm 36 is preferably about 4.0 mm of travel.

Received into body 22 is a grip 40 having a second longitudinal bore 42 therethrough. An acceptable grip 40 for use with the present invention is a one-half inch threaded style stainless steel grip, item number STTUB-S, sold by WorldWide Tattoo Supply, a division of TCM Supply Corp., 15410 Stafford St., Industry, Calif. 91744, U.S.A. A barrel 44 of grip 40 is received into a yoke 46 of body 22, and a thumbscrew 48 of body 22 tightens onto the barrel 44, thereby permitting grip 40 to be adjustable to a plurality of positions, e.g., 50 and 52, closer to and further away with respect to body 22 in a manner hereinafter described.

Grip 40 also includes an adapter 54 that is threadedly received into the threaded distal end 56 of grip 40. Adapter 54 has a well-known male Luer lock 58 on its distal end 60 and the proximal end 62 of adapter 54 is threaded with a 5/16-32M thread to match the threaded distal end 56 of grip 40. A suitable adapter 54 for use with grip 40 is the part number VN536 adapter sold by Vita Needle Company, 919 Great Plain Ave., Needham, Mass. 02492, U.S.A.

First needle 24 is preferably a well-known 21 gauge hollow hypodermic needle having a well-known Luer fitting 64 and safety covering 66 thereon, with Luer fitting 64 being twistingly and securely received into Luer lock 58 of adapter 54. A suitable safety needle assembly for first needle 24 is the Monoject Magellan safety needle, a 21G×⅝ inch needle assembly, part number 8881850158, sold by Kendall Healthcare, a division of Covidien PLC, 15 Hampshire St., Mansfield, Mass., 02048, U.S.A.

Second needle 28 is preferably a 26 gauge by 3.5 inch (8.9 cm) stainless steel spinal needle such as the B-Braun Spinocan spinal needle, product code S2635, reference number 333300, sold by B. Braun Medical, Inc., USA, 824 Twelfth Ave., Bethlehem, Pa. 18018-3524, U.S.A.

Apparatus 20 further preferably includes a linkage 68, such as bar 70 having a circular loop 72 at its proximal end 74 that is received onto crankarm 36 as hereinafter described, with the distal end 76 of bar 70 being fixedly and substantially coaxially attached, as by soldering, to the proximal end 78 of second needle 28.

To assemble apparatus 20, the distal tip 80 of second needle 28 is inserted into the longitudinal bore 42 through the barrel 44 of grip 40 until the distal tip 80 of second needle 28 protrudes from the threaded distal end 56 of grip 40. The distal tip 80 of second needle 28 is then inserted into the threaded proximal end 62 of adapter 54 until distal tip 80 extends from the Luer lock 58 on the distal end 60 of adapter 54. The threaded proximal end 62 of adapter 54 is then threadedly inserted into the threaded distal end 56 of grip 40, and adapter 54 is then securely tightened onto grip 40. The distal tip 80 of second needle 28 is then inserted into longitudinal bore 26 through the distal end 82 of first needle 24, and Luer fitting 64 is twistingly engaged with and securely received into Luer lock 58 of adapter 54.

A rubber bushing grommet 84 is then inserted into circular loop 72 at the proximal end 74 of bar 70, the proximal end 74 of bar 70 and barrel 44 of grip 40 is inserted through the yoke 46 of body 22 as the central hole 86 of grommet 84 is pushed onto crankarm 36, and thumbscrew 48 of body 22 is then tightened onto the barrel 44, thereby fixedly mounting first needle 24 to body 22.

By loosening thumbscrew 48, barrel 44 of grip 40 can be pushed into and pulled out of yoke 46 and then retightened after adjustment to one of a plurality of positions, e.g., 50 and 52, closer to and further away with respect to body 22, thereby allowing the distance 88 between the distal end 82 of first needle 24 and body 22 to be adjusted so that the distal tip 80 of second needle 28 maximally extends a distance 90 of about 2.5 to 3.0 mm beyond the distal end 82 of first needle 24 during each reciprocation stroke.

The method of using the needle aponeurotomy apparatus 20 of the present invention is to provide an assembled apparatus 20 as heretofore described, and then to loosen thumbscrew 48 and adjust grip 40 as heretofore described so that the distal tip 80 of second needle 28 maximally extends about 2.5 to 3.0 mm beyond the distal end 82 of first needle 24 during each reciprocation stroke. The doctor then sticks the first needle 24 into a patient's hand H and causes the motor means to reciprocate the second needle with respect to the body. The doctor then directs the apparatus 20 so that the second needle 28 is caused to repeatedly penetrate a Dupuytren's band of the patient's hand, thereby weakening the Dupuytren's band. The doctor then forcibly extends the contracted finger, causing the Dupuytren's band to “snap” and break.

### INDUSTRIAL APPLICABILITY

The present invention is a needle aponeurotomy apparatus and method of use for performing needle aponeurotomy upon a Dupuytren's band in a patient's hand, having a faster reciprocation speed than heretofore seen in the prior art and having a repeatable reciprocation stroke of the aponeurotomy needle that has not been heretofore known.

Although the present invention has been described and illustrated with respect to a preferred embodiment and a preferred use therefor, it is not to be so limited since modifications and changes can be made therein which are within the full intended scope of the invention.

We claim:

1: A needle aponeurotomy apparatus for performing needle aponeurotomy upon a Dupuytren's band in a patient's hand, said apparatus comprising:
(a) a handheld body;
(b) a first needle having a longitudinal bore therethrough, said first needle being fixedly mounted to said body and having a distal end;
(c) a second needle received into said bore, said needle having a distal tip;
(d) motor means for reciprocating said second needle with respect to said body such that said tip of said second needle reciprocatingly maximally extends to a certain distance beyond said distal end of said first needle during each reciprocation.

2: The apparatus as recited in claim 1, in which said apparatus further comprises a linkage interposed between said motor means and said second needle, said linkage having a first end coupled to said second needle and having a second end for being moved by said motor means, such that movement of said second end of said linkage causes said first end of said linkage to reciprocate said second needle within said bore.

3: The apparatus as recited in claim 1, in which said body includes a grip, said grip including a Luer lock into which said first needle is fixedly received, said grip being adjustable to a plurality of positions closer to and further away with respect to said body such that a distance between said distal end of said first needle and said body may be adjusted.

4: A method of performing needle aponeurotomy upon a Dupuytren's band in a patient's hand, said method comprising the steps of:
(a) providing a needle aponeurotomy apparatus as recited in claim 3;
(b) adjusting said grip so that said tip of said second needle maximally extends about 2.5 to 3.0 mm beyond said distal end of said first needle during each reciprocation of said second needle;
(c) sticking said first needle into a patient's hand and causing said motor means to reciprocate said second needle with respect to said body; and then
(d) causing said second needle to repeatedly penetrate a Dupuytren's band of said patient.

5: A method of performing needle aponeurotomy upon a Dupuytren's band in a patient's hand, said method comprising the steps of:
(a) providing a needle aponeurotomy apparatus as recited in claim 1;
(b) sticking said first needle into a patient's hand and causing said motor means to reciprocate said second needle with respect to said body; and then
(c) causing said second needle to repeatedly penetrate a Dupuytren's band of said patient.
